(19) United States
(12) Patent Application Publication
(10) Pub. No.: US 2024/0139493 A1
(43) Pub. Date: May 2, 2024

(54) Disinfecting Device Containing Chlorhexidine-Based Solution

(71) Applicant: Becton, Dickinson and Company, Franklin Lakes, NJ (US)

(72) Inventors: Kevin M. Ryan, Whitehouse Station, NJ (US); Andrew Woodbridge Van Cott, Hillsborough, NC (US); Richard Bradley Timmers, Westwood, NJ (US); Shoshana San Solo, New York, NY (US); Shishir Prasad, Ramsey, NJ (US); Narasinha C. Parasnis, Nanuet, NY (US); Anurag Mathur, Philadelphia, PA (US); Chang Jiang, Yonkers, NY (US); Kelsey Ann Graves, Baltimore, MD (US); Joseph J. Dajcs, Fort Worth, TX (US); Qin Chen, Parsippany, NJ (US)

(21) Appl. No.: 17/979,165

(22) Filed: Nov. 2, 2022

(51) Int. Cl. A61M 39/16

(52) U.S. Cl. CPC A61M 39/162

(57) ABSTRACT

Provided herein is a disinfecting device including a container containing a chlorhexidine-based solution having a chlorhexidine concentration of from 0.2-2.0% wt/vol. Provided also is a medical connector disinfected with the disinfecting device. A method of disinfecting a medical connector using the disinfecting device is also disclosed.

### BACKGROUND OF THE INVENTION

### Field of the Invention

The present invention is directed to a disinfecting device containing a chlorhexidine-based solution.

### Description of Related Art

Disinfecting devices may be used to disinfect medical connectors (e.g., intravenous catheter connectors) before use on patients. Disinfecting devices can include disinfecting caps or scrubbing devices. Isopropyl alcohol (IPA) is commonly used as the disinfecting material in these disinfecting devices. However, while the IPA suitably disinfects the connector, it evaporates in seconds leaving no persistent disinfecting material on the connector. The connector can, therefore, become contaminated after evaporation of the IPA, leaving the connector at risk for contamination prior to use.

### SUMMARY OF THE INVENTION

Provided herein is a disinfecting device including a container containing a chlorhexidine-based solution having a chlorhexidine concentration of from 0.2-2.0% wt/vol.

Also provided herein is a disinfected medical connector including a medical connector disinfected with the disinfecting device described herein.

Also provided herein is a method of disinfecting a medical connector including contacting the medical connector in a chlorhexidine-based solution comprising a chlorhexidine concentration of from 0.2-2.0% wt/vol contained in a container of a disinfecting device.

In accordance with an embodiment of the present invention, a disinfecting device having a container containing a chlorhexidine-based solution including a chlorhexidine concentration of from 0.2-2.0% wt/vol.

In accordance with an embodiment of the present invention, the chlorhexidine concentration is from 0.4-1.5% wt/vol or from 0.5-1.0% wt/vol.

In accordance with an embodiment of the present invention, the chlorhexidine-based solution comprises chlorhexidine acetate and/or chlorhexidine gluconate.

In accordance with an embodiment of the present invention, the chlorhexidine-based solution includes isopropyl alcohol.

In accordance with an embodiment of the present invention, the chlorhexidine-based solution includes an isopropyl alcohol concentration of at least 25% wt/vol or at least 50% wt/vol.

In accordance with an embodiment of the present invention, the disinfecting device includes a disinfecting cap or a disinfecting scrubbing device.

In accordance with an embodiment of the present invention, the disinfecting device is configured to engage with a medical connector.

In accordance with an embodiment of the present invention, the medical connector includes a needle-free connector, a male connector, and/or a stop cock.

In accordance with an embodiment of the present invention, the disinfecting device is sterilized.

In accordance with an embodiment of the present invention, the disinfecting device is sterilized using steam sterilization, an autoclave, and/or radiation sterilization.

In accordance with an embodiment of the present invention, the chlorhexidine-based solution includes an oil.

In accordance with an embodiment of the present invention, a disinfected medical connector includes a medical connector disinfected with the disinfecting device as described herein.

In accordance with an embodiment of the present invention, the disinfecting device includes a disinfecting cap engaged with the medical connector.

In accordance with an embodiment of the present invention, the disinfecting device includes a scrubbing device containing the chlorhexidine-based solution into which the medical connector is submerged.

In accordance with an embodiment of the present invention, a method of disinfecting a medical connector including contacting the medical connector in a chlorhexidine-based solution including a chlorhexidine concentration of from 0.2-2.0% wt/vol contained in a container of a disinfecting device.

In accordance with an embodiment of the present invention, the chlorhexidine-based solution includes an isopropyl alcohol concentration of at least 25% wt/vol or at least 50% wt/vol.

In accordance with an embodiment of the present invention, sterilizing the disinfecting device occurs by using steam sterilization, an autoclave, and/or radiation sterilization.

In accordance with an embodiment of the present invention, a disinfecting solution includes a chlorhexidine-based solution comprising a chlorhexidine concentration of from 0.2-2.0% wt/vol.

In accordance with an embodiment of the present invention, the chlorhexidine concentration is from 0.4-1.5% wt/vol or from 0.5-1.0% wt/vol.

In accordance with an embodiment of the present invention, the chlorhexidine-based solution comprises chlorhexidine acetate and/or chlorhexidine gluconate.

In accordance with an embodiment of the present invention, the chlorhexidine-based solution includes isopropyl alcohol.

In accordance with an embodiment of the present invention, the chlorhexidine-based solution includes an isopropyl alcohol concentration of at least 25% wt/vol or at least 50% wt/vol.

In accordance with an embodiment of the present invention, the chlorhexidine-based solution includes an oil.

### BRIEF DESCRIPTION OF THE DRAWINGS

FIG. 1 is a schematic view of a disinfecting device according to non-limiting embodiments described herein;

FIG. 2 is a schematic view of a disinfecting device as a cap according to non-limiting embodiments described herein; and

FIG. 3 is a schematic view of a disinfecting device as a scrubbing device according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

The present disclosure is directed to a disinfecting device comprising a container containing a chlorhexidine-based solution comprising a chlorhexidine concentration of from 0.2-2.0% wt/vol.

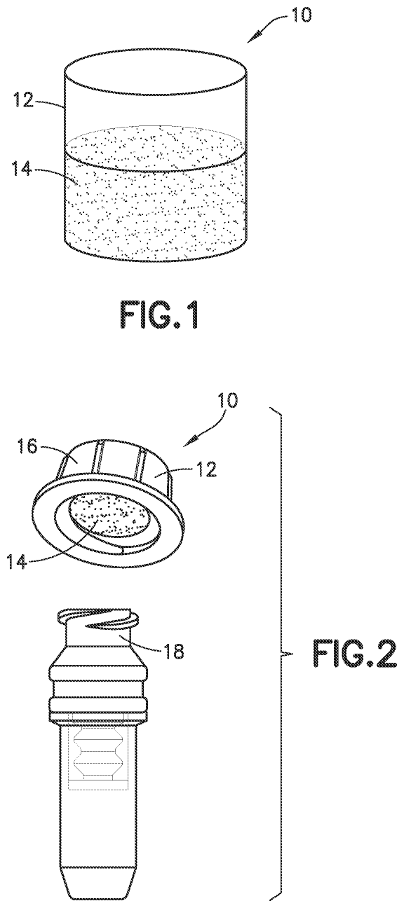

Referring to FIGS. 1-3, a disinfecting device 10 may comprise a container 12. The container 12 may contain a chlorhexidine-based (CH-based) solution 14 (a disinfecting solution). The CH-based solution may be an aqueous solution (e.g., comprise water).

The CH-based solution 14 may comprise a CH. The CH may have a concentration of from 0.2-2.0% wt/vol, such as from 0.4-1.5% wt/vol or from 0.5-1.0% wt/vol. Concentrations of CH within these ranges may provide persistent disinfecting effect, while being low enough in concentration to be safe for use with patients (e.g., entering a patient's bloodstream) and to not interfere with the engagement between the disinfecting device 10 and a connector 18 engaged therewith. A persistent disinfecting effect may be achieved due to CH having a higher molecular weight and a lower evaporation rate than other solvents suitable for use in disinfecting devices 10, such as isopropyl alcohol (IPA).

The CH in the CH-based solution 14 may comprise chlorhexidine acetate (CHA), chlorhexidine gluconate (CHG), chlorhexidine hydrochloride, or some combination thereof.

The CH-based solution 14 may be an aqueous solution of CH. The CH-based solution 14 may further comprise a disinfecting agent as a solvent for the CH-based solution 14. The disinfecting agent solvent in the CH-based solution 14 may comprise IPA, aqueous povidone iodide solution, or any other suitable disinfecting agent. The CH-based solution 14 may comprise a disinfecting solvent (e.g., IPA) concentration of at least 25% wt/vol or at least 50% wt/vol or at least 70% wt/vol. The presence of the aqueous solvent and/or the disinfecting solvent in the CH-based solution 14 may aid in forming a uniform CH layer on the surface of the connector 18, resulting in a persistent disinfected surface.

To confirm that the presence of CH in the disinfecting solution (in combination with the IPA) does not impact the antimicrobial performance of the disinfecting device compared to a disinfecting device containing only IPA, a study was conducted with two challenge microorganisms (one Gram-positive and one Gram-negative bacteria) and two CH variants (CHA and CHG) at concentrations ranging from 0.1% to 2.0%. As can be seen from Table 1 below, the presence of CH does not adversely impact the antimicrobial performance of the disinfecting device.

TABLE 1

| | | | | | Percent Log | |
|---|---|---|---|---|---|---|
| Test | | | Log Reduction | | Reduction | |
| Article | Microorganism | Baseline | 15 Sec | 30 Sec | 15 Sec | 30 Sec |
| 70% IPA | Pseudomonas aeruginosa | 7.63 | 7.63 | 7.63 | 100% | 100% |
| | Staphylococcus aureus | 7.90 | 7.90 | 7.90 | 100% | 100% |
| 70% IPA | Pseudomonas aeruginosa | 7.63 | 7.63 | 7.63 | 100% | 100% |
| with 0.1% | Staphylococcus aureus | 7.90 | 7.90 | 7.90 | 100% | 100% |
| CHA | | | | | | |
| 70% IPA | Pseudomonas aeruginosa | 7.63 | 7.63 | 7.63 | 100% | 100% |
| with 2.0% | Staphylococcus aureus | 7.90 | 7.90 | 7.90 | 100% | 100% |
| CHA | | | | | | |
| 70% IPA | Pseudomonas aeruginosa | 7.63 | 7.63 | 7.63 | 100% | 100% |
| with 0.1% | Staphylococcus aureus | 7.90 | 7.90 | 7.90 | 100% | 100% |
| CHG | | | | | | |
| 70% IPA | Pseudomonas aeruginosa | 7.63 | 7.63 | 7.63 | 100% | 100% |
| with 0.5% | Staphylococcus aureus | 7.90 | 7.90 | 7.90 | 100% | 100% |
| CHG | | | | | | |
| 70% IPA | Pseudomonas aeruginosa | 7.63 | 7.63 | 7.63 | 100% | 100% |
| with 2.0% | Staphylococcus aureus | 7.90 | 7.90 | 7.90 | 100% | 100% |
| CHG | | | | | | |

The persistent disinfection performance of the CH-based solution 14 was tested on caps for IV catheters. Non-limiting examples of the CH-based solution 14 were prepared using a 70% IPA and 0.5% CHG (wt/vol %) solution and a 0.5% aqueous CHG solution (wt/vol %) and used to treat caps for IV catheters. These CH-based solutions were compared to caps for IV catheters that were untreated or caps for IV catheters treated with 70% IPA (wt/vol %). The results of the disinfection of the caps using the various solutions is shown in Table 2 below.

TABLE 2

| | Test 1 | | Repeat of Test 1 | |
|---|---|---|---|---|
| | Recovered | | Recovered | |
| | Log₁₀ | | Log₁₀ | |
| | CFU/Device | | CFU/Device | |
| Disinfecting | (mean ± | Log | (mean ± | Log |
| Caps (N = 5) | stdev) | Reduction | stdev) | Reduction |
| Untreated | 6.06 ± 0.21 | — | 6.40 ± 0.14 | — |
| 70% IPA | 5.99 ± 0.23 | 0.07 ± 0.23 | — | — |
| (wt/vol %) | | | | |
| 70% IPA and | 4.46 ± 0.38 | 1.60 ± 0.38 | 4.72 ± 0.54 | 1.45 ± 0.54 |
| 0.5% CHG | | | | |
| (wt/vol %) | | | | |
| 0.5% aqueous | 5.52 ± 0.23 | 0.54 ± 0.23 | 5.93 ± 0.37 | 0.47 ± 0.37 |
| CHG solution | | | | |
| (wt/vol %) | | | | |

The results in Table 2 show that since IPA evaporates fast, no persistent disinfection performance was observed using only the IPA solution. An average of 0.5 log₁₀ CFU was observed for the 0.5% aqueous CHG solution. Mixing 0.5% CHG with 70% IPA resulted in an average of approximately 1.5 log₁₀ CFU reduction. The improved log₁₀ CFU reduction in the solutions containing CHG indicated an improved persistent disinfection, which can be attributed to the formation of a CHG coating on the IV connector surface.

The CH-based solution 14 may further comprise an oil. Non-limiting embodiments of oils that may be suitable for use in the CH-based solution 14 comprise plant-derived oils, such as cottonseed oils, eucalyptus oils, grapeseed oils, cajeput oils or some combination thereof. The oil may aid disengagement between the disinfecting device 10 and the connector 18 by decreasing the removal force to disengage the components.

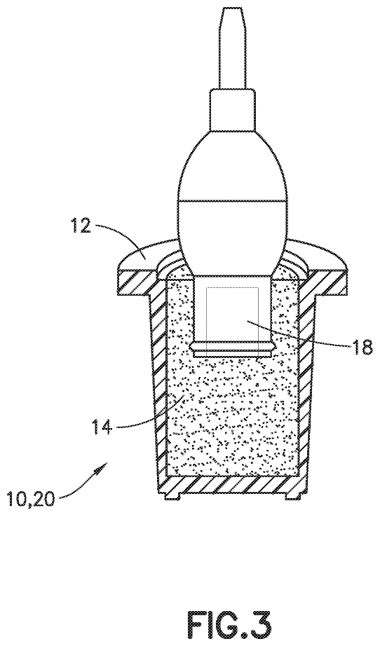

Referring to FIGS. 2 and 3, the disinfecting device 10 may comprise a disinfecting cap 16 or a disinfecting scrubbing device 20.

FIG. 2 shows a non-limiting example of the disinfecting device 10 as the disinfecting cap 16. The disinfecting cap 16 may engage with the connector 18 and may function as a cap over an open end of the connector 18. The disinfecting cap 16 may engage with the connector 18 in any suitable matter. For example, the disinfecting cap 16 may screw onto the connector 18 to disinfect the connector 18 by the connector 18 being submerged in the CH-based solution 14 contained in the container 12 of the disinfecting cap 16. A non-limiting example of a suitable cap that may modified to contain the CH-based solution 14 and function as the disinfecting device 10/disinfecting cap 16 is the BD PureHub which may be modified to contain the CH-based solution 14 described herein.

FIG. 3 shows a non-limiting example of the disinfecting device 10 as the disinfecting scrubbing device 20. The connector 18 may be submerged in the CH-based solution 14 contained in the container 12 of the disinfecting scrubbing device 20 to disinfect the connector 18.

The disinfecting device 10 may additionally be sterilized. The disinfecting device 10 may be sterilized using steam sterilization. The disinfecting device 10 may be sterilized using an autoclave. The disinfecting device 10 may be sterilized using radiation sterilization, such as using E-beam, X-ray, and/or Gamma sterilization. The dose range for Gamma and E-beam radiation applied to the disinfecting device 10 may range from 10-90 kGy, such as from 12-80 kGy, or from 15-60 kGy. Sterilization of the disinfecting device 10 may be performed to kill any microbes for which the CH-based solution 14 is less effective.

With continued reference to FIGS. 2 and 3, the disinfecting device 10 may be configured to engage with the connector 18, such as a medical connector (e.g., an intravenous catheter connector). The connector 18 may comprise a needle-free connector, a male connector, and/or a stop cock. Non-limiting, commercial examples of connectors for which the disinfection device 10 may be configured to engage are the BD SmartSite and Q-Syte needle-free connectors. However, it will be appreciated that the design of the disinfecting device 10 may be adapted to engage any type of connector used in the medical field that is commonly disinfected before being used on a patient.

With continued reference to FIGS. 2 and 3, the present disclosure is also directed to a disinfected medical connector. The disinfected medical connector may comprise the connector 18 disinfected with the disinfecting device 10 described herein. The connector 18 may be disinfected to form the disinfected medical connector by engaging the connector 18 with the disinfecting cap 16 described herein containing the CH-based solution 14. The connector 18 may be disinfected to form the disinfected medical connector by submerging the connector 18 in the scrubbing device 20 described herein containing the CH-based solution 14.

The present disclosure is also directed to a method of disinfecting the medical connector 18. The connector 18 may be disinfected by contacting the connector 18 in the CH-based solution 14 of the disinfecting device 10, the CH-based solution 14 comprising the chlorhexidine concentration of from 0.2-2.0% wt/vol contained in 12 container of the disinfecting device 10. The CH-based solution 14 may comprise an IPA concentration of at least 25% wt/vol or at least 50% wt/vol or at least 70% wt/vol. The method may comprise sterilizing the disinfecting device 10 using steam sterilization, an autoclave, and/or radiation sterilization. Submerging the connector 18 in the CH-based solution 14 may comprise engaging the connector 18 with the disinfecting cap 16. Submerging the connector 18 in the CH-based solution 14 may comprise submerging the connector 18 in the CH-based solution 14 contained in the container 12 of the disinfecting scrubbing device 20.

The connector 18 may be submerged in the CH-based solution 14 for at least 5 seconds, such as at least 8 seconds, or at least 10 seconds to sufficiently disinfect the connector 18.

Although the present disclosure has been described in detail for the purpose of illustration based on what is currently considered to be the most practical and preferred embodiments, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

The invention claimed is:

1. A disinfecting device comprising a container containing a chlorhexidine-based solution comprising a chlorhexidine concentration of from 0.2-2.0% wt/vol.

2. The disinfecting device of claim 1, wherein the chlorhexidine concentration is from 0.4-1.5% wt/vol or from 0.5-1.0% wt/vol.

3. The disinfecting device of claim 1, wherein the chlorhexidine-based solution comprises chlorhexidine acetate and/or chlorhexidine gluconate.

4. The disinfecting device of claim 1, wherein the chlorhexidine-based solution comprises isopropyl alcohol.

5. The disinfecting device of claim 4, wherein the chlorhexidine-based solution comprises an isopropyl alcohol concentration of at least 25% wt/vol or at least 50% wt/vol.

6. The disinfecting device of claim 1, wherein the disinfecting device comprises a disinfecting cap or a disinfecting scrubbing device.

7. The disinfecting device of claim 1, wherein the disinfecting device is configured to engage with a medical connector.

8. The disinfecting device of claim 7, wherein the medical connector comprises a needle-free connector, a male connector, and/or a stop cock.

9. The disinfecting device of claim 1, wherein the disinfecting device is sterilized.

10. The disinfecting device of claim 9, wherein the disinfecting device is sterilized using steam sterilization, an autoclave, and/or radiation sterilization.

11. A disinfected medical connector comprising:
a medical connector disinfected with the disinfecting device of claim 1.

12. The disinfected medical connector of claim 11, wherein the disinfecting device comprises a disinfecting cap engaged with the medical connector.

13. The disinfected medical connector of claim 11, wherein the disinfecting device comprises a scrubbing device containing the chlorhexidine-based solution into which the medical connector is submerged.

14. A method of disinfecting a medical connector comprising contacting the medical connector in a chlorhexidine-based solution comprising a chlorhexidine concentration of from 0.2-2.0% wt/vol contained in a container of a disinfecting device.

15. The method of claim 14, wherein the chlorhexidine-based solution comprises an isopropyl alcohol concentration of at least 25% wt/vol or at least 50% wt/vol.
